(19) United States
(12) Plant Patent Application Publication
(10) Pub. No.: US 2002/0188982 P1
(43) Pub. Date: Dec. 12, 2002

(54) Chrysanthemum plant named 'Gedi One Gal'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,921

(22) Filed: Jun. 4, 2001

(51) Int. Cl. A01H 5/00

(52) U.S. Cl. PLT/293

(57) ABSTRACT

A new and distinct Chrysanthemum plant variety characterized by a large decorative type inflorescence, consistent flowering response to short days, blooming consistently after 49 days of short day length, free branching habit, and dark reddish-purple ray florets, which resist fading with age.

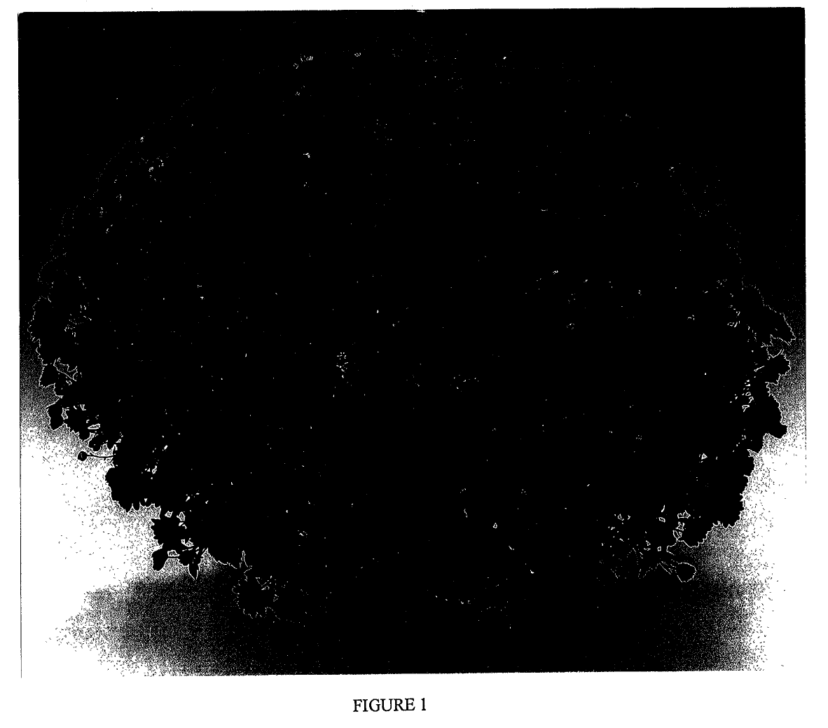

### BACKGROUND OF THE INVENTION

The present invention relates to a new and distinct cultivar of Chrysanthemum plant, botanically known as Dendranthema morifolium and herein after referred to by the cultivar name ‘GEDI ONE GAL.’

This new cultivar was the result of hybridization of a female parent, ‘Tripoli’ (U.S. Plant Pat. No. 10,207) with a male parent, ‘Ampiro’ (unpatented in the United States). The new cultivar was selected in October 1997.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE GAL’ has not been observed under all possible environmental conditions. The phenotype may vary somewhat with variations in environment such as temperature, daylength, and light intensity, without, however, any variance in genotype.

The following traits have been repeatedly observe and are determined to be the unique characteristics of ‘GEDI ONE GAL.’ These characteristics in combination distinguish ‘GEDI ONE GAL’ as a new and distinct Chrysanthemum:

1. Large decorative type inflorescence;

2. Consistent flowering response to short days, blooming consistently after 49 days of short day length;

3. Free branching habit; and

4. Dark redish-purple ray florets, resisting fading with age.

Plants of the new Chrysanthemum are similar to plants of the parent cultivars in most horticultural characteristics, however plants of the new cultivar differ from plants of the female parent cultivar ‘Tripoli’ in the following characteristics:

1. The new cultivar has a different bloom type;

2. The new cultivar has many more ray florets than the parent; and

3. The new cultivar has individual blooms resisting fading better.

The new cultivar also differed from the male parent ‘Ampiro’ in the following characteristics:

1. The new cultivar has a wider diameter flower; and

2. The new cultivar has different color ray florets.

The new cultivar can be compared to the Chrysanthemum cultivar ‘Jenny Wren’ (disclosed in Plant Pat. No. 10,213). However plants of the new Chrysanthemum differed from the plants of the cultivar ‘Jenny Wren’ in the following characteristics:

1. The new cultivar naturally blooms 2 weeks later;

2. The new cultivar has darker reddish-purple ray florets;

3. The new cultivar has a more round plant shape;

4. The new cultivar has many more ray florets; and

5. The new cultivar has ray florets which are resistant to fading.

### BRIEF DESCRIPTION OF THE PHOTOGRAPHS

The photograph shows a plant of ‘GEDI ONE GAL’ grown in a 2-gallon container. A single cutting was used in the pot, planted mid-May and grown under outdoor field conditions.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema mnorifolium cultivar ‘GEDI ONE GAL’

Commercial classification: Garden-type Chrysanthemum

Parentage: Hybridization of female parent ‘Tripoli’ (U.S. Plant Pat. No. 10,207) and male parent ‘Ampiro ’ (unpatented in the United States)

### Ppropagation

Type: Asexual vegetative

Time to Rooting: 7 to 14 days at approximately 21° C.

Rooting habit: Fine, fibrous

### Plant

Growth Habit: Mounding herbaceous perennial

Height: Approximately 28 cm

Growth rate: Moderate

Branching Characteristics: Free Branching

Length of Lateral Branches: Approximately 23 cm

Number of leaves per lateral branch: Approximately 28

Stem color: Near 137A

### Foliage

Leaf:

Arrangement.—Alternate single

Average Length.—Approximately 7.8 cm

Average Width.—Approximately 5.2 cm

Shape of blade.—Ovate

Apex.—Acuminate

Base.—Attenuate

Attachment.—Stalked

Margin.—Serrate

Texture of top surface.—Lightly pubescent

Texture of bottom surface.—Lightly pubescent

Color.—young foliage upper side: Near 137A young foliage under side: Near 137B mature foliage upper side: Near 137A mature foliage under side: Near 137B venation upper side: Near 138C venation under side: Near 137C

Petiole:

Average length.—Approximately 3.0 cm

### Bloom

Inflorescence:

Flowering habit.—Induced by darkness period greater than 13.5 hours, approximately 49 days of appropriate day length required to induce and develop blooms

Inflorescence form.—Decorative

Natural flowering season.—Approximately the last week in September, first week in October

Number of inflorescences per stem.—Approximately 15

Inflorescence diameter.—Approximately 8.0 cm

Inflorescence depth.—2.2 cm

Inflorescence longevity on plant.—Approximately 24-29 days

Persistence.—Persistent

Ray Florets:

Appearance.—Matte

Texture.—Smooth

Average number per flower.—270

Shape.—Oblanceolate

Margin.—Entire

Apex.—Obtuse

Length.—Approximately 3.9 cm

Width.—Approximately 1 cm

Color.—upper surface at first opening: Near 59A upper surface at maturity: Near 59B upper surface at fading: Near 53B under surface at first opening: Near 53A under surface at maturity: Near 53B under surface at fading: Near 53B

Disc florets:

Appearance.—Shiny

Texture.—Smooth

Average number per flower.—Approximately 40

Shape.—Cylindric

Apex.—Obtuse

Average length.—Approximately 1 cm

Average width.—Approximately 0.2 cm

Color.—at first opening: Near 2A at maturity: Near 21 A at fading: Near 17D

Peduncle:

Length.—at terminal end (shortest): Approximately 7 cm at lateral end (longest): Approximately 11 cm

Strength.—Moderate

Color.—Near 137C

Habit.—Erect

Inflorescence bud:

Length.—Approximately 1 cm

Diameter.—Approximately 1 cm

Form.—Globular

Color.—Near 59A

Sepals:

Appearance.—Matte

Texture.—Lightly pubescent

Number.—Approximately 60 to 70

Shape.—Ovate

Margin.—Entire

Apex.—Acute

Color.—upper side: Near 137A under side: Near 137A

### Reproductive Organs

Ray Florets:

Pistillate.—Number of pistils per flower: 1 Stigma shape: 2 branched Stigma color: Near 7A Style color: Near 16D Style length: Approximately 0.5 cm

Disc Florets:

Pistillate.—Number of pistils per flower: 1 Stigma shape: Cylindric Style length: Approximately 0.5 cm

Staminate.—Number of stamens per flower: Approximately 5 Anther shape: Tubular Anther color: Near 6A Pollen color: Near 7B

### Other Characteristics

Disease Resistance: Neither resistance nor susceptibility observed

‘GEDI ONE GAL’: Table of R.H.S. Color Codes

| | | | |
|---|---|---|---|
| FOLIAGE | Leaf | young foliage upper surface | Near 137A |
| | | young foliage under surface | Near 137B |
| | | mature foliage upper surface | Near 137A |
| | | mature foliage under surface | Near 137B |
| | | venation upper surface | Near 138C |
| | | venation under surface | Near 137C |
| BLOOM | Ray Florets | upper surface at first opening | Near 59A |
| | | upper surface at maturity | Near 59B |
| | | upper surface at fading | Near 53B |
| | | under surface at first opening | Near 53A |
| | | under surface at maturity | Near 53B |
| | | under surface at fading | Near 53B |
| | | Pistillate: Stigma | Near 7A |
| | | Pistillate: Style | Near 16D |
| | Disc Florets | at first opening | Near 2A |
| | | at maturity | Near 21A |
| | | at fading | Near 17D |
| | | Staminate: Anther | Near 6A |
| | | Staminate: Pollen | Near 7B |
| | Peduncle | | Near 137C |
| | Inflorescence | | Near 59A |
| | Bud | | |
| | Sepals | upper surface | Near 137A |
| | | under surface | Near 137A |

What is claimed is:

1. A new and distinct variety of Chrysanthemum plant named ‘GEDI ONE GAL’ as herein illustrated and described.
